(19) United States
(12) Patent Application Publication
(10) Pub. No.: US 2009/0239941 A1
(43) Pub. Date: Sep. 24, 2009

(54) AMINO ACID DERIVATIVES

(75) Inventor: Christopher Hobbs, London (GB)

Correspondence Address: BANNER & WITCOFF, LTD., 1100 13th STREET, N.W., SUITE 1200, WASHINGTON, DC, 20005-4051, US

(73) Assignee: PROXIMAGEN LTD., London (GB)

(21) Appl. No.: 12/282,913

(22) PCT Filed: Mar. 13, 2007
(86) PCT No.: PCT/GB2007/000860

§ 371 Date: Mar. 5, 2009

(30) Foreign Application Priority Data
Mar. 16, 2006 (GB) 0605295.5

(51) Int. Cl. A61K 31/216; C07C 69/017; C07C 69/612; C07D 339/04; A61K 31/385; A61P 25/00

(52) U.S. Cl. 514/440; 560/193; 560/85; 549/39; 514/548; 514/533

(57) ABSTRACT

Compounds of formula (I) are active as dopaminergic compounds or as compounds which or as compounds which diminish the symptoms of dopamine deficiency: wherein: R₁ and R₂ are independently selected from —C(═O)R₅ or —C(═O)OR₅; or one of R₁ and R₂ is hydrogen and the other is —C(═O)R₅ or —C(═O)OR₅; R₃ and R₄ are independently selected from hydrogen, optionally substituted C₁-C₆ alkyl, C₃-C₆ cycloalkyl, C₂-C₆ alkenyl, or C₂-C₆ alkynyl, —CH2Q, —C(═O)R₅, —C(═O)OR₅, —C(═O)NR₅R₆, or R₅ is hydrogen or optionally substituted C₁-C₆ alkyl or —CH₂Q; R₆ is hydrogen or optionally substituted C₁-C₆ alkyl Or —CH₂Q; and Q is an optionally substituted monocyclic carbocyclic or heterocyclyl ring of 3 to 6 ring atoms.

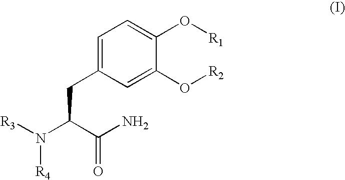

The present invention relates to compounds which diminish the symptoms of dopamine deficiency.

Dopamine is a substance produced naturally by neurons in the basal ganglia of the brain that allows smooth, coordinated control of voluntary movement. Loss of, or impairment of, dopamine-producing neurons in the brain is implicated in Parkinson's disease and related Parkinson-plus syndromes. These conditions respond to dopamine replacement therapy. Other conditions, for example, Restless Legs Syndrome (RLS) also respond to dopamine replacement therapy.

Parkinson's disease is a progressive neurodegenerative disorder that affects neuronal cells in the substantia nigra in the mid-brain. It is an age-related disorder of the central nervous system primarily attacking people over the age of 60. Approximately one out of every 500 people contract the illness and approximately one out of every 100 people over the age of 60 develop the illness. As indicated above, Parkinson's disease is thought to be caused by a deficiency of dopamine. The common symptoms include tremor, stiffness (or rigidity) of muscles, slowness of movement (bradykinesia) and loss of balance (postural dysfunction). Parkinson's Disease is one of the most prevalent neurodegenerative illnesses. The natural history of the disease is progressive and from 10-15 years from onset of the disease becomes disabling in most patients.

Parkinson's disease is largely sporadic and referred to as idiopathic in nature. Forms of the illness due to vascular incidents and to toxin exposure also exist. Rare familial forms of the illness also exist.

Many treatments have been tried since James Parkinson first described the condition in 1817. Current therapy for Parkinson's disease is based on dopamine replacement therapy based on the use of the dopamine precursor levodopa (or L-dopa) or dopaminergic compounds. L-dopa is highly effective in reversing the motor symptoms of the illness but on chronic treatment and with disease progression, its effectiveness declines. The duration of drug response is reduced and unpredictable fluctuations in movement occur. Treatment is associated with therapy limiting side effects which include involuntary movements (dyskinesia) and psychosis.

RLS is a neurosensorimotor disorder with parestethesias, sleep disturbances and, in most cases, periodic limb movements of sleep (PLMS). Two forms of RLS appear to exist: the idiopathic and the uremic form. RLS is characterised by (1) a desire to move the legs, usually associated with paresthesias/dysesthesias, (2) motor restlessness, (3) worsening or exclusive presence of symptoms at rest (i.e. lying, sitting) with at least partial or temporary relief by activity, and (4) worsening of symptoms during the evening or night.

The present invention provides compounds which are active as dopaminergic compounds or as compounds which or as compounds which diminish the symptoms of dopamine deficiency.

According to the invention, there is provided a compound of formula (I) or a salt, hydrate or solvate thereof:

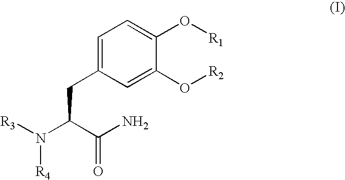

wherein:
R₁ and R₂ are independently selected from —C(═O)R₅ or —C(═O)OR₆; or one of R₁ and R₂ is hydrogen and the other is —C(═O)R₅ or —C(═O)OR₅;
R₃ and R₄ are independently selected from
hydrogen,
optionally substituted C₁-C₆ alkyl, C₃-C₆ cycloalkyl C₂-C₆ alkenyl, or C₂-C₈ alkynyl,
—CH₂Q,
—C(═O)R₅,
—C(═O)OR₅,
—C(═O)NR₆R₆, or
   R₅ is hydrogen or optionally substituted C₁-C₆ alkyl or —CH₂Q;
   R^{G} is hydrogen or optionally substituted C₁-C₆ alkyl or —CH₂Q; and
   Q is an optionally substituted monocyclic carbocyclic or heterocyclyl ring of 3 to 6 ring atoms;
   PROVIDED THAT when R₁ and R₂ are each —C(═O)R₅ wherein R₅ is methyl, and R₃ is hydrogen, then R₄ is not tert-butoxycarbonyl.

The proviso in the above definition of the compounds of the invention excludes a compound disclosed in Int. J. Peptide and Protein Res. (1979), 14(3), 234-46. However, that publication discloses no pharmaceutical utility for the compound.

As used herein, the term “(Cₐ-C_{b})alkyl” wherein a and b are integers refers to a straight or branched chain alkyl radical having from a to b carbon atoms. Thus when a is 1 and b is 6, for example, the term includes methyl, ethyl, n-propyl, isopropyl, n-butyl, isobutyl, sec-butyl, t-butyl, n-pentyl and n-hexyl.

As used herein the term “(Cₐ-C_{b})alkenyl” means a straight or branched chain alkenyl moiety having from a to b carbon atoms having at least one double bond of either E or Z stereochemistry where applicable. Thus when a is 2 and b is 6, the term includes, for example, vinyl, allyl, 1- and 2-butenyl and 2-methyl-2-propenyl.

As used herein the term “C₂-C₆ alkynyl” refers to straight chain or branched chain hydrocarbon groups having from a to b carbon atoms and having in addition one triple bond. Thus when a is 2 and b is 6, the term includes, for example, ethynyl, 1-propynyl, 1- and 2-butynyl, 2-methyl-2-propynyl, 2-pentynyl, 3-pentynyl, 4-pentynyl, 2-hexynyl, 3-hexynyl, 4-hexynyl and 5-hexynyl.

As used herein the unqualified term “carbocyclic” refers to a mono-, bi- or tricyclic radical having up to 16 ring atoms, all of which are carbon, and includes aryl and cycloalkyl.

As used herein the unqualified term “cycloalkyl” refers to a monocyclic saturated carbocyclic radical having from 3-8 carbon atoms and includes, for example, cyclopropyl, cyclobutyl, cyclopentyl, cyclohexyl, cycloheptyl and cyclooctyl.

As used herein the unqualified term “aryl” refers to a mono-, bi- or tri-cyclic carbocyclic aromatic radical, and includes radicals having two monocyclic carbocyclic aromatic rings which are directly linked by a covalent bond. Illustrative of such radicals are phenyl, biphenyl and napthyl.

As used herein the unqualified term “heteroaryl” refers to a mono-, bi- or tri-cyclic aromatic radical containing one or more heteroatoms selected from S, N and O, and includes radicals having two such monocyclic rings, or one such monocyclic ring and one monocyclic aryl ring, which are directly linked by a covalent bond. Illustrative of such radicals are thienyl, benzthienyl, furyl, benzfuryl, pyrrolyl, imidazolyl, benzimidazolyl, thiazolyl, benzthiazolyl, isothiazolyl, benzisothiazolyl, pyrazolyl, oxazolyi, benzoxazolyl, isoxazolyl, benzisoxazolyl, isothiazolyl, triazolyl, benztriazolyl, thiadiazolyl, oxadiazolyl, pyridinyl, pyridazinyl, pyrimidinyl, pyrazinyl, triazinyl, indolyl and indazolyl.

As used herein the unqualified term “heterocyclyl” or “heterocyclic” includes “heteroaryl” as defined above, and in its non-aromatic meaning relates to a mono-, bi- or tri-cyclic non-aromatic radical containing one or more heteroatoms selected from S, N and O, and to groups consisting of a monocyclic non-aromatic radical containing one or more such heteroatoms which is covalently linked to another such radical or to a monocyclic carbocyclic radical. Illustrative of such radicals are pyrrolyl, furanyl, thienyl, piperidinyl, imidazolyl, oxazolyl, isoxazolyl, thiazolyl, thiadiazolyl, pyrazolyl, pyridinyl, pyrrolidinyl, pyrimidinyl, morpholinyl, piperazinyl, indolyl, morpholinyl, benzfuranyl, pyranyl, isoxazolyl, benzimidazolyi, methylenedioxyphenyl, ethylenedioxyphenyl, maleimido and succinimido groups.

Unless otherwise specified in the context in which it occurs, the term “substituted” as applied to any moiety herein means substituted with up to four compatible substituents, each of which independently may be, for example, (C₁-C₆)alkyl, (C₃-C₆) cycloalkyl, (C₁-C₆)alkoxy, hydroxy, hydroxy(C₁-C₆)alkyl, mercapto, mercapto(C₁-C₆)alkyl, (C₁-C₆)alkylthio, phenyl, monocyclic heterocyclic, benzyl, phenoxy, halo (including fluoro, bromo and chloro), trifluoromethyl, trifluoromethoxy, nitro, nitrile —CN), oxo, —COOH, —COOR^{A}, —COR^{A}, —SO₂R^{A}, —CONH₂, —SO₂NH₂, —CONHR^{A}, —SO₂NHR^{A}, —CONR^{A}R^{B}, —SO₂NR^{A}R^{B}, —NH₂—, —NHR^{A}, —NR^{A}R^{B}, —OCONH₂, —OCONHR^{A}—OCONR^{A}R^{B}, —NHCOR^{A}, —NHCOOR^{A}, —NR^{B}COOR^{A}, —NHSO₂OR^{A}, —NR^{B}SO₂OH, —NR^{B}SO₂OR^{A}, —NHCONH₂, —NR^{A}CONH₂, —NHCONHR^{B}—NR^{A}CONHR^{B}, —NHCONR^{A}R^{B}, or —NR^{A}CONR^{A}R^{B} wherein R^{A} and R^{B} are independently a (C₁-C₆)alkyl, (C₃-C₆) cycloalkyl, phenyl, benzyl or monocyclic heterocyclic having 5 or 6 ring atoms, or R^{A} and R^{B} when attached to the same nitrogen may, together with that nitrogen, form a 4- to 6-membered ring containing that nitrogen. An “optional substituent” may be one of the foregoing substituent groups.

As used herein the term “salt” includes base addition, acid addition and quaternary salts. Compounds of the invention which are acidic can form salts, including pharmaceutically acceptable salts, with bases such as alkali metal hydroxides, e.g. sodium and potassium hydroxides; alkaline earth metal hydroxides e.g. calcium, barium and magnesium hydroxides; with organic bases e.g. N-methyl-D-glucamine, choline tris(hydroxymethyl)amino-methane, L-arginine, L-lysine, N-ethyl piperidine, dibenzylamine and the like. Those compounds (I) which are basic can form salts, including pharmaceutically acceptable salts with inorganic acids, e.g. with hydrohalic acids such as hydrochloric or hydrobromic acids, sulphuric acid, nitric acid or phosphoric acid and the like, and with organic acids e.g. with acetic, tartaric, succinic, fumaric, maleic, malic, salicylic, citric, methanesulphonic, p-toluenesulphonic, benzoic, benzenesunfonic, glutamic, lactic, and mandelic acids and the like.

In the compounds of the invention, carbon atom to which R1 is attached is assymmetric, and the stereochemistry at that centre is as shown in formula (I). However, the compounds of the invention may contain one or more additional chiral centres, because of the presence of asymmetric carbon atoms, and they can exist as a number of diastereoisomers with R or S stereochemistry at each chiral centre. The invention includes all such diastereoisomers and mixtures thereof.

### The Groups R₁ and R₂

One of R₁ and R₂ may be hydrogen, but presently it is preferred that R₁ and R₂ are independently —C(═O)R₅. Furthermore it is presently preferred that R₁ and R₂ be the same —C(═O)R₅. For example, R₁ and R₂ may be the same —C(═O)R₅ wherein R₅ is ethyl, isopropyl, 2,2-dimethylpropyl, 4-methylphenyl or, preferably, methyl.

### The Groups R₃ and R₄

R₃ and R₄ are independently selected from
hydrogen,
optionally substituted C₁-C₆ alkyl, C₃-C₆ cylcoalkyl, C₂-C₆ alkenyl, or C₂-C₆ alkynyl, for example methyl, ethyl, allyl, or propargyl;
—CH₂Q wherein Q is an optionally substituted monocyclic carbocyclic or heterocyclyl ring of 3 to 6 ring atoms, for example phenyl, 2- or 3-thienyl, 2- or 3-furanyl, 2- or 3 pyrrolyl, 2-, 3- or 4-pyridyl, cyclopropyl, cyclopentyl, or cyclohexyl;
—C(═O)R₅ wherein R₅ is hydrogen or, preferably, optionally substituted C₁-C₆ alkyl, for example methyl, ethyl, n- or isopropyl, or —CH₂Q as discussed above.
—C(═O)NR₅R₆, wherein R₅ and R₆ are each preferably hydrogen, but one or both of R₅ and R₆ may also be optionally substituted C₁-C₆ alkyl, for example methyl, ethyl, n- or isopropyl, or —CH₂Q as discussed above; or
—C(═O)OR₅ wherein R₅ may be optionally substituted C₁-C₆ alkyl, for example methyl, ethyl, n- or isopropyl, or —CH₂Q as discussed above. It is presently preferred that R₅ be ethyl.

### Optional Substituents in any of R₁-R₆

Any optional substituents in the groups R₁-R₆ may be selected from, for example, methyl, trifluoromethyl, methoxy, trifluoromethoxy, cyclopropyl, halogen, cyano, hydroxy, mercapto, oxo, —NH₂, —NHR^{A}, or —NR^{A}R^{B} wherein R^{A} and R^{B} are independently methyl or ethyl.

Examples of specific compounds of the invention include those of the examples herein.

### Synthetic Routes

There are multiple synthetic strategies for the synthesis of the compounds (I) with which the present invention is concerned, but all rely on chemistry known to the synthetic organic chemist. Compounds according to formula (I) can be synthesised according to procedures described in the standard literature and are well-known to the one skilled in the art. Typical literature sources are “Advanced organic chemistry”, 4^{th} Edition (Wiley), J March, “Comprehensive Organic Transformation”, 2^{nd} Edition (Wiley), R. C. Larock, “Handbook of Heterocyclic Chemistry”, 2^{nd} Edition (Pergamon), A. R. Katritzky), review articles such as found in “Synthesis”, “Acc. Chem. Res.”, “Chem. Rev”, or primary literature sources identified by standard literature searches online or from secondary sources such as “SciFinder” or “Beilstein”.

For example, some of the compounds of the invention are accessible by introduction of the groups R₃ and/or R₄ onto the non-protected non-amido amino group of compounds (IA)

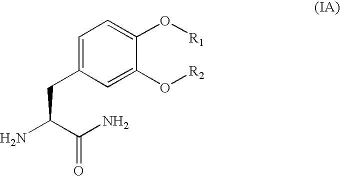

Other compounds of the invention are accessible by introduction of the R₁ and R₂ groups onto the free hydroxyl groups of a compound (IB)

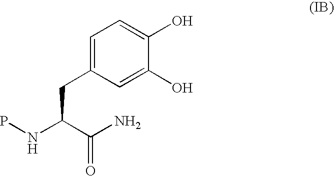

wherein P is a protected amino group, followed by conversion of the protected amino group to a free amino group. Of course, where one of R₁ and R₂ in compounds (I) is to be hydrogen, one of the hydroxy groups in (IB) may be protected while the R₁ or R₂ group is introduced onto the other, and that protected hydroxyl group may subsequently be deprotected.

The Examples herein provide examples of the synthetic routes which may be employed for synthesis of the compounds of the invention.

### Pharmaceutical Utility

The compounds of the present invention are useful in a method of treatment of a condition associated with impaired dopaminergic signalling in a subject, comprising administering to the subject an amount of the compound effective to reduce such impairment. The compounds are also useful in the preparation of a composition for treatment of a condition associated with impaired dopaminergic signalling. Examples of such conditions include Parkinson's disease, or Restless Legs Syndrome, as well as Tourette's syndrome, attention deficit hyperactive disorder, generation of pituitary tumours, a Parkinson-plus syndrome, levodopa responsive dystonia, dyskinesia, periodic movements in sleep, dysphagia or neuroleptic malignant syndrome.

Examples of Parkinson's disease which can be treated with the compounds of the invention include sporadic Parkinson's disease, familial forms of Parkinson's disease and post-encephalitic Parkinsonism.

Examples of Parkinson-plus syndromes which can be treated with the compounds of the invention include progressive supranuclear palsy and multiple system atrophy.

The dyskinesia may be L-dopa-induced dyskinesia.

The compounds of the invention may be administered in a variety of dosage forms. Thus, they can be administered orally, for example as tablets, capsules, troches, lozenges, aqueous or oily suspensions, dispersible powders or granules.

The compounds can be administered in a sublingual formulation, for example a buccal formulation. The compounds of the invention may also be administered parenterally, whether subcutaneously, intravenously, intramuscularly, intrasternally, transdermally, by inhalation (e.g. intranasally) or by infusion techniques. The compounds may also be administered as suppositories. Typically, the compounds of the invention are administered orally or by inhalation (e.g. intranasally). Preferably, the compounds of the invention are administered orally. More preferably, the compounds of the invention are administered as a tablet or capsule.

The present invention further provides a pharmaceutical composition containing a compound of formula (I), or a pharmaceutically acceptable salt thereof, as defined above, and a pharmaceutically acceptable carrier.

The compounds of the invention are typically formulated for administration with a pharmaceutically acceptable carrier or diluent. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non toxic and pharmacologically inactive substances used in pharmaceutical formulations. Such pharmaceutical preparations may be manufactured in known manner, for example, by means of mixing, granulating, tableting, sugar coating, or film coating processes.

Liquid dispersions for oral administration may be syrups, emulsions and suspensions. The syrups may contain as carriers, for example, saccharose or saccharose with glycerine and/or mannitol and/or sorbitol. Suspensions and emulsions may contain as carrier, for example a natural gum, agar, sodium alginate, pectin, methylcellulose, carboxymethylcellulose, or polyvinyl alcohol. The suspension or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Since the compounds of the invention are preferably administered orally, the present invention further provides a pharmaceutical composition containing a compound of formula (I), or a pharmaceutically acceptable salt thereof, as defined above, and a pharmaceutically acceptable carrier in the form of a capsule or tablet.

Solutions for injection or infusion may contain as carrier, for example, sterile water or preferably they may be in the form of sterile, aqueous, isotonic saline solutions.

The compounds of the present invention may also be administered with a peripheral decarboxylase inhibitor. The present invention therefore provides a pharmaceutical composition containing a compound of formula (I), or a pharmaceutically acceptable salt thereof, as defined above, a peripheral decarboxylase inhibitor and a pharmaceutically acceptable carrier or diluent. Typically the peripheral decarboxylase inhibitor is carbidopa or benserazide. Preferably the peripheral decarboxylase inhibitor is carbidopa.

Also provided is a product comprising (a) a compound of the formula (I), or a pharmaceutically acceptable salt thereof, as defined above and (b) a peripheral decarboxylase inhibitor as defined above, for simultaneous separate or sequential use in the treatment of the human or animal body.

Further, said medicament is typically for co-administration with a peripheral decarboxylase inhibitor defined above.

It will be understood that the specific dose level for any particular patient will depend upon a variety of factors including the activity of the specific compound employed, the age, body weight, general health, sex, diet, time of administration, route of administration, rate of excretion, drug combination and the severity of the particular disease undergoing treatment. Optimum dose levels and frequency of dosing will be determined by clinical trial. However, it is expected that a typical dose will be in the range from about 0.001 to 50 mg per kg of body weight.

The following examples illustrate the invention:

### HPLC/MS Method A

The system used to obtain LC-MS data comprised an HP1100 LC combined with a Waters Micromass ZMD mass spectrometer operating in positive ion mode.

ZMD Mass Spectrometer

| | | | |
|---|---|---|---|
| Capillary | 3.5 kV | Cone | 30 V |
| Extractor | 3 V | Source Temp | 100° C. |
| Desolvation Temp | 220° C. | Cone Gas | 58 L/Hr |
| Desolvation Gas | 350 L/Hr | Multiplier | 750 V |

Data were acquired in a full scan from 150 to 800 m/z

| | |
|---|---|
| Scan duration | 1.0 s |
| Interscan delay | 0.1 s |

### HPLC

The reverse phase separation was carried out on a Genesis C18 column (50×3.2 mm with 4 μm silica).

| Injection Volume | 10 | μL |
|---|---|---|
| UV data | 210 to 400 | nm |
| Sample Temperature | ambient | |
| Column Temperature | ambient | |
| Flow Rate | 1.0 | mL/min |
| Split to ZMD | 0.33 | mL/min |

LC Method

| | |
|---|---|
| Solvent A | Water/0.1% formic acid |
| Solvent B | CH₃CN/0.1% formic acid |

Gradient Program for LC Method

| Time | A % | B % | C % | D % | Flow | Curve |
|---|---|---|---|---|---|---|
| 0.00 | 95.0 | 5.0 | 0.0 | 0.0 | 1.0 | 6 |
| 7.00 | 5.0 | 95.0 | 0.0 | 0.0 | 1.0 | 6 |
| 10.00 | 5.0 | 95.0 | 0.0 | 0.0 | 1.0 | 6 |
| 14.00 | 95.0 | 5.0 | 0.0 | 0.0 | 1.0 | 6 |

### HPLC/MS Method B

The system used to obtain LC-MS data comprised a Waters Alliance 2695 quaternary HPLC, Waters 996 Photo Diode Array (PDA) detector and Waters ZQ 2000 single quadrupole mass spectrometer. The ZQ can acquire data simultaneously in positive and negative electrospray ionisation modes.

ZQ Mass Spectrometer

| | | | |
|---|---|---|---|
| Capillary | 3.3 kV/−3.0 kV | Cone | 40 V/−40 V |
| Extractor | 5 V/−5 V | Source Temp | 110° C. |
| Desolvation Temp | 400° C. | Cone Gas | 40 L/Hr |
| Desolvation Gas | 350 L/Hr | Multiplier | 500 V/−500 V |

Data were acquired in a full scan from 80 to 1000 m/z

| | |
|---|---|
| Scan duration | 0.80 s |
| Interscan delay | 0.20 s |

### HPLC

The reverse phase separation was carried out on a Zorbax XDB C8 column (150×4.6 mm with 5 μm silica from Agilent).

| | | |
|---|---|---|
| Injection Volume | 10 | μL |
| UV data | 220 to 400 | nm |
| Sample Temperature | 20° | C. |
| Column Temperature | 30° | C. |
| Flow Rate | 1.0 | mL/min |
| Split to ZQ | 0.3 | mL/min |

LC Method (approximately pH 3.2)

| | |
|---|---|
| Solvent A | Water/10 mM NH₄HCO₂/0.1% formic acid |
| Solvent B | 95% CH₃CN/5% A/0.1% formic acid |

Gradient Program for LC Method

| Time | A % | B % | C % | D % | Flow | Curve |
|---|---|---|---|---|---|---|
| 0.00 | 95.0 | 5.0 | 0.0 | 0.0 | 1.000 | 1 |
| 1.00 | 95.0 | 5.0 | 0.0 | 0.0 | 1.000 | 6 |
| 11.00 | 5.0 | 95.0 | 0.0 | 0.0 | 1.000 | 6 |
| 14.20 | 5.0 | 95.0 | 0.0 | 0.0 | 1.000 | 6 |
| 14.50 | 95.0 | 5.0 | 0.0 | 0.0 | 1.000 | 6 |
| 15.00 | 95.0 | 5.0 | 0.0 | 0.0 | 1.000 | 6 |

### Abbreviations

### DMA N,N-Dimethylacetamide

### DMAP 4-N,N-Dimethylaminopyridine

### DMF N,N-Dimethylformamide

EDCI 3-Dimethylaminopropyl-N-ethylcarbodiimide hydrochloride
HBTU Benzotriazolyl N,N,N,N-tetramethyluronium hexafluorophosphate

### EXAMPLE 1

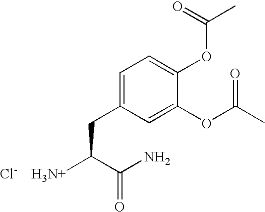

### (S)-1-Carbamoyl-2-(3,4-diacetoxyphenyl)-ethylammonium chloride

### Step 1

### (S)-3-(3,4-Bis-benzyloxyphenyl)-2-tert-butoxycarbonylaminopropionic acid methyl ester

(S)-2-tert-Butoxycarbonylamino-3-(3,4-dihydroxyphenyl)-propionic acid methyl ester (14.5 g) was dissolved in acetone (250 ml). Potassium carbonate (20 g) and potassium iodide (1.1 g) were added followed by benzyl chloride (13 ml). The reaction mixture was stirred at room temperature under nitrogen for 15 min and then heated to reflux for 16 hr. Benzyl chloride (3 ml) was then added and reflux continued until completion of reaction (hplc).

The solvent was removed by evaporation under reduced pressure and the residue partitioned between ethyl acetate and water. The ethyl acetate extract was washed with brine, dried (MgSO₄) and evaporated to dryness giving an off-white solid. Recrystallisation from diisopropyl ether (S)-3-(3,4-bis-benzyloxyphenyl)-2-tert-butoxy-carbonylaminopropionic acid methyl ester as a colourless solid, 14.9 g; HPLC/MS (Method A) retention time 7.23 min, m/z 392 (MH⁺-Boc).

### Step 2

### (S)-3-(3,4-Bis-benzyloxyphenyl)-2-tert-butoxycarbonylaminopropionic acid

(S)-3-(3,4-Bis-benzyloxyphenyl)-2-tert-butoxycarbonylaminopropionic acid methyl ester (12.03 g) was suspended in methanol (250 ml). 2M Lithium hydroxide (16 ml) was added dropwise with stirring under nitrogen and the mixture then stirred at room temperature overnight. More 2M lithium hydroxide (2 ml) was added and the mixture stirred at 45 C for 45 min. The resulting solution was concentrated and partitioned between 2M HCl (40 ml) and ethyl acetate. The extract was washed with water and brine, dried (MgSO₄) and evaporated to give 3(S)-3-(3,4-bis-benzyloxyphenyl)-2-tert-butoxycarbonylaminopropionic acid as a colourless solid, 11.7 g; HPLC/MS (Method A) retention time 6.61 min, m/z 378 (MH⁺-Boc).

### Step 3

### [(S)-2-(3,4-Bis-benzyloxyphenyl)-1-carbamoylethyl]-carbamic acid tert-butyl ester

(S)-3-(3,4-Bis-benzyloxyphenyl)-2-tert-butoxycarbonylaminopropionic acid (4.8 g) was dissolved in dry DMA (35 ml). HBTU (4.6 g) was added with stirring. After 30 min a solution of ammonia in dioxane (0.5M, 40 ml) was added and stirring continued for 16 hr. Additional HBTU (3 g) was added to the reaction mixture followed by ammonia solution (0.5M, 20 ml).

After 2 hr the mixture was partitioned between water and ethyl acetate. The ethyl acetate extract was washed with brine and water, dried (MgSO₄) and evaporated to dryness. The colourless solid was recrystallised from ethyl acetate to give [(S)-2-(3,4-Bis-benzyloxyphenyl)-1-carbamoylethyl]-carbamic acid tert-butyl ester, 3.5 g; HPLC/MS (Method A) retention time 6.82 min, m/z 477 (MH⁺).

### Step 4

### [(S)-1-Carbamoyl-2-(3,4-dihydroxyphenyl)-ethyl]-carbamic acid tert-butyl ester

[(S)-2-(3,4-Bis-benzyloxyphenyl)-1-carbamoylethyl]-carbamic acid tert-butyl ester (3.65 g) was dissolved in a mixture of methanol (120 ml) and dioxane (60 ml). A suspension of 5% Pd/C (360 mg) in methanol (10 ml) was added and the solution stirred overnight under an atmosphere of hydrogen gas.

The catalyst was removed by filtration through celite and evaporated to dryness giving [(S)-1-Carbamoyl-2-(3,4-dihydroxyphenyl)-ethyl]-carbamic acid tert-butyl ester, 2.15 g; HPLC/MS (Method A) retention time 3.69 min, m/z 297 (MH⁺).

### Step 5

### Acetic acid 2-acetoxy-4-((S)-2-tert-butoxycarbonylamino-2-carbamoylethyl)-phenyl ester

[(S)-1-Carbamoyl-2-(3,4-dihydroxyphenyl)-ethyl]-carbamic acid tert-butyl ester (356 mg) was suspended in dry dichloromethane (5 ml) and DMA (1 ml). Acetyl chloride (0.22 ml) was added followed by triethylamine (0.42 ml) and a catalytic amount of DMAP. The mixture was stirred and heated at 50 C overnight.

The reaction mixture was diluted with dichloromethane and washed with dil HCl, aq NaHCO₃ and with brine. Drying (MgSO₄) and evaporation gave the crude product which was recrystallised from ethyl acetate-hexane. Acetic acid 2-acetoxy-4-((S)-2-tert-butoxycarbonylamino-2-carbamoylethyl)-phenyl ester was obtained as a colourless solid, 304 mg; HPLC/MS (Method A) retention time 4.76 min, m/z 381 (MH⁺), 398 (MNH₄⁺).

### Step 6

### (S)-1-Carboxy-2-(3,4-diacetoxy-phenyl)-ethylammonium chloride

Acetic acid 2-acetoxy-4-((S)-2-tert-butoxycarbonylamino-2-carbamoyl-ethyl)-phenyl ester (298 mg) was dissolved in 4M HCl dioxane (6 ml). The solution was stirred at room temperature and a colourless precipitate slowly formed.

After approximately 1.5 hr ether (6 ml) was added and the colourless solid was collected by filtration, washed with ether and dried in vacuo at ca 40° C.

(S)-1-Carboxy-2-(3,4-diacetoxy-phenyl)-ethylammonium chloride was obtained as a colourless solid, 243 mg; HPLC/MS (Method B) retention time 4.32 min, m/z 281 (MH⁺); NMR (500 MHz, d₆ DMSO) 2.26 (3H, s), 2.28 (3H, s), 3.03 (1H, dd J 14, 7.5), 3.13 (1H, dd J 14, 6), 3.98 (1H, dd J 7, 6), 7.18 (1H, d J 2), 7.20 (1H, dd J 8.5, 2), 7.23 (1H, d J 8.5), 7.58 (1H, br s), 7.99 (1H, br s), 8.26 (3H, br s, exch D₂O);

### EXAMPLE 2

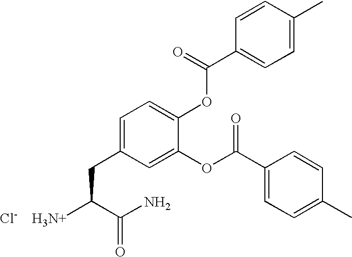

### (S)-2-[3,4-Bis-(4-methylbenzoyloxy)-phenyl]-1-carbamoylethylammonium chloride

### Step 1

### 4-Methyl-benzoic acid 4-((S)-2-tert-butoxycarbonylamino-2-carbamoylethyl)-2-(4-methylbenzoyloxy)-phenyl ester

[(S)-1-Carbamoyl-2-(3,4-dihydroxy-phenyl)-ethyl]-carbamic acid tert-butyl ester (444 mg) was dissolved in dry DMA (15 ml) under nitrogen. p-Toluoyl chloride (579 mg) was added with stirring followed by triethylamine (378 mg) and a catalytic amount of DMAP. The reaction mixture was stirred overnight at room temperature and then diluted with water (40 ml). The precipitated solid was filtered of, washed with water and dried. The crude solid was purified by silica gel chromatography eluting with ethyl acetate-dichloromethane (2:1). 4-Methyl-benzoic acid 4-((S)-2-tert-butoxycarbonylamino-2-carbamoylethyl)-2-(4-methylbenzoyloxy)-phenyl ester was obtained as a colourless solid, 520 mg; HPLC/MS (Method A) retention time 7.18 min; m/z 533 (MH⁺), 550 (MNH₄⁺).

### Step 2

### (S)-2-[3,4-Bis-(4-methylbenzoyloxy)-phenyl]-1-carboxyethylammonium chloride

4-Methyl-benzoic acid 4-((S)-2-tert-butoxycarbonylamino-2-carbamoylethyl)-2-(4-methylbenzoyloxy)-phenyl ester (520 mg) was added in portions to a stirred solution of HCl in dioxane (4M, 10 ml). After approximately 1.5 hr the solution was evaporated to dryness giving a colourless foam. Addition of ether gave (S)-2-[3,4-bis-(4-methylbenzoyloxy)-phenyl]-1-carboxyethylammonium chloride as a colourless solid, 387 mg; HPLC/MS (Method B) retention time 7.81 min, m/z 433 (MH⁺); NMR (500 MHz, d₆ DMSO) 2.35 and 2.36 (together 6H, two s), 3.11 (1H, dd J 14.5, 8), 3.21 (1H, dd J 14.5, 6), 4.06 (1H, br, collapse to dd with D₂O), 7.27-7.33 (5H, m), 7.42 (1H, d J 2), 7.46 (1H, d J 8), 7.62 (1H br s), 7.83-7.87 (4H, m), 8.02 (1H, br s), 8.31 (3H, br s, exch D₂O).

### EXAMPLE 3

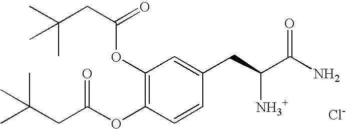

### (S)-2-[3,4-Bis-(3,3-dimethylbutyryloxy)-phenyl]-1-carbamoylethylammonium chloride

### Step 1

### 3,3-Dimethyl-butyric acid 4-((S)-2-tert-butoxycarbonylamino-2-carbamoylethyl)-2-(3,3-dimethylbutyryloxy)-phenyl ester

[(S)-1-Carbamoyl-2-(3,4-dihydroxyphenyl)-ethyl]-carbamic acid tert-butyl ester (444 mg) was treated with t-butylacetyl chloride in DMA as described in example 4 step 1. 4-3,3-Dimethyl-butyric acid 4-((S)-2-tert-butoxycarbonylamino-2-carbamoylethyl)-2-(3,3-dimethylbutyryloxy)-phenyl ester was obtained as a colourless solid, 670 mg; HPLC/MS (Method A) retention time 7.48 min, m/z 493 (MH⁺), 510 (MNH₄⁺).

### Step 2

### (S)-2-[3,4-Bis-(3,3-dimethylbutyryloxy)-phenyl]-1-carbamoylethylammonium chloride

3,3-Dimethylbutyric acid 4-((S)-2-tert-butoxycarbonylamino-2-carbamoyl-ethyl)-2-(3,3-dimethylbutyryloxy)-phenyl ester (650 mg) was treated with 4M HCl-dioxane (10 ml) as described in example 4 step 2.

(S)-2-[3,4-Bis-(3,3-dimethylbutyryloxy)-phenyl]-1-carbamoylethylammonium chloride was obtained as an off-white solid, 510 mg; NMR (500 MHz, de DMSO) 1.06 and 1.07 (together ca 18H, two s), 2.43 and 2.44 (together ca 4H, two s), 3.02 (1H, dd J 14, 8), 3.13 (1H, dd J 14, 5.5) 3.98 (1H, br t), 7.17-7.22 (3H, m) 7.59 (1H, br s), 7.98 (1H, br s) 8.24 (3H, br s exch D₂O); HPLC/MS (Method B) retention time 8.15 min, m/z 393 (MH⁺).

### EXAMPLE 4

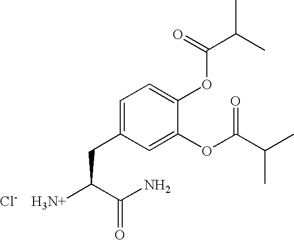

### (S)-2-(3,4-Bisisobutyryloxyphenyl)-1-carbamoylethylammonium chloride

### Step 1

### Isobutyric acid 4-((S)-2-tert-butoxycarbonylamino-2-carbamoylethyl)-2-isobutyryloxy-phenyl ester

[(S)-1-Carbamoyl-2-(3,4-dihydroxyphenyl)-ethyl]-carbamic acid tert-butyl ester (376 mg) was treated with isobutyryl chloride and triethylamine in DMA as described in example 4 step 1. Excess acid chloride and base were added and the mixture was heated to 70° C. overnight in order to complete the reaction. Isobutyric acid 4-((S)-2-tert-butoxycarbonylamino-2-carbamoylethyl)-2-isobutyryloxyphenyl ester was obtained as a colourless solid, 105 mg; HPLC/MS (Method A) retention time 6.25 min, m/z 437 (MH⁺), 454 (MNH₄⁺).

### Step 2

### (S)-2-(3,4-Bis-isobutyryloxyphenyl)-1-carbamoylethylammonium chloride

Isobutyric acid 4-((S)-2-tert-butoxycarbonylamino-2-carbamoylethyl)-2-isobutyryloxy-phenyl ester (105 mg) was dissolved in 4M HCl-dioxane (3 ml) at room temperature. After approximately 1.5 hr solvent was evaporated to give a gum that was triturated with ether to give (S)-2-(3,4-bis-isobutyryloxyphenyl)-1-carbamoylethylammonium chloride as a colourless solid, 85 mg; NMR (500 MHz, d₆ DMSO) 1.20 and 1.22 (together 12H, two d J ca 7), 2.78 and 2.80 (together 2H, two sept J ca 7), 3.02 (1H, dd J 14, 7.5), 3.12 (1H, dd J 14, 6) 3.98 (1H, dd J 7.5, 6), 7.18-7.20 (2H, m, shows d J 2 and dd J 8, 2 in D₂O), 7.23 (1H, d J 8), 7.60 (1H, br s), 7.94 (1H, br s), 8.21 (3H, br s exch D₂O); HPLC/MS (Method B) retention time 6.49 min, m/z 337 (MH⁺).

### EXAMPLE 5

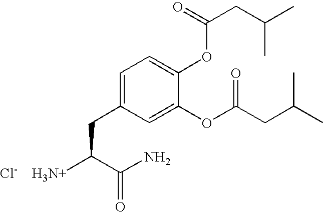

### (S)-2-[3,4-Bis-(3-methylbutyryloxy)-phenyl]-1-carbamoylethylammonium chloride

### Step 1

### 3-Methyl-butyric acid 4-((S)-2-tert-butoxycarbonylamino-2-carbamoylethyl)-2-(3-methylbutyryloxy)-phenylester

[(S)-1-Carbamoyl-2-(3,4-dihydroxyphenyl)-ethyl]-carbamic acid tert-butyl ester (350 mg) was treated with isovaleryl chloride and triethylamine in DMA as described in example 4 step 1. 3-Methylbutyric acid 4-((S)-2-tert-butoxycarbonylamino-2-carbamoylethyl)-2-(3-methylbutyryloxy)-phenylester was obtained as a colourless solid, 224 mg; HPLC/MS (Method A) retention time 6.95 min, m/z 465 (MH⁺), 454 (MNH₄⁺).

### Step 2

### (S)-2-[3,4-Bis-(3-methylbutyryloxy)-phenyl]-1-carbamoylethylammonium chloride

3-Methyl-butyric acid 4-((S)-2-tert-butoxycarbonylamino-2-carbamoylethyl)-2-(3-methylbutyryloxy)-phenylester (224 mg) was treated with 4M HCl-dioxane (5 ml) as described in example 4 step 2. Evaporation of solvent and addition of methanol and ethergave (S)-2-[3,4-bis-(3-methylbutyryloxy)-phenyl]-1-carbamoylethylammonium chloride as a pale yellow solid, 84 mg; NMR (500 MHz, d₆ DMSO) 0.98 and 0.99 (together 12H, two d J 7), 2.01-2.11 (2H, m), 2.43 and 2.44 (together 4H, two d J 7), 3.02 (1H, dd J 14, 7.5), 3.13 (1H, dd J 14, 6), 3.98 (1H, dd J 7, 6), 7.18-7.23 (3H, m), 7.59 (1H, br s), 7.97 (1H, br s), 8.23 (3H, br s exch D₂O); HPLC/MS (Method B) retention time 7.54 min, m/z 365 (MH⁺).

### EXAMPLE 6

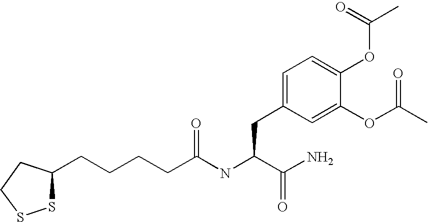

### Acetic acid 2-acetoxy-4-[(S)-2-carbamoyl-2-((R)-5-[1,2]dithiolan-3-yl-pentanoylamino)-ethyl]-phenyl ester

(S)-1-Carbamoyl-2-(3,4-diacetoxyphenyl)-ethylammonium chloride (0.316 g) was dissolved in dichloromethane (10 mL)/DMF(2 mL). Triethylamine (0.121 g) was added, followed by the addition of HOBt (0.149 g) and R(+)-α-lipoic acid. The mixture was stirred at room temperature for 20 min. EDC (0.211 g) was added and stirring was continued at room temperature overnight. The reaction mixture was diluted with dichloromethane (20 mL), then washed with water, brine, and dried over sodium sulfate. After removal of the solvent, the residue was purified by silica gel chromatography eluting with ethyl acetate/methanol (9.5:0.5). Acetic acid 2-acetoxy-4-[(S)-2-carbamoyl-2-((R)-5-[1,2]dithiolan-3-yl-pentanoylamino)-ethyl]-phenyl ester was obtained as a yellow solid, 0.380 g. m/z 469 (MH⁺), 491 (MNa⁺). ¹H NMR (360 MHz, CDCl₃) δ 1.37-1.50 (m, 2H), 1.58-1.70 (m, 4H), 1.85-1.94 (m, 1H), 2.21 (t, J=7.4, 2H), 2.27 (s, 3H), 2.28 (s, 3H), 2.41-2.49 (m, 1H), 2.98 (m, 1H), 3.07-3.21 (m, 3H), 3.52-3.59 (m, 1H), 4.63-4.69 (m, 1H), 5.47 (s, 1H), 5.95 (s, 1H), 6.26 (d, J=7.7 Hz, 1H), 7.08 (s, 1H), 7.12 (s, 2H).

### EXAMPLE 7

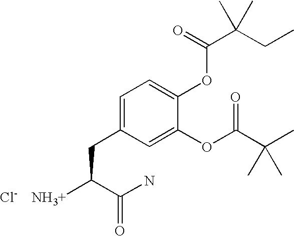

### (S)-2-[3,4-Bis-(2,2-dimethyl-butyryloxy)-phenyl]-1-carbamoyl-ethyl-ammonium chloride

### Step 1

### 2,2-Dimethyl-butyric acid 4-((S)-2-tert-butoxycarbonylamino-2-carbamoyl-ethyl)-2-(2,2-dimethyl-butyryloxy)-phenyl ester

[(S)-1-Carbamoyl-2-(3,4-dihydroxy-phenyl)-ethyl]-carbamic acid tert-butyl ester (0.50 g) was suspended in dry dichloromethane (12 mL). Triethylamine (0.393 g) was added and the mixture was cooled with in an ice-bath. 2,2-dimethylbutanoyl chloride (0.50 g) was added dropwise. The mixture was stirred at 0° C. for 2 hours, washed with 10% sodium bicarbonate, brine and dried over sodium sulfate. After removal of the solvent, the residue was purified by silica gel chromatography eluting with ethyl acetate/hexane (1:1). 2,2-Dimethyl-butyric acid 4-((S)-2-tert-butoxycarbonylamino-2-carbamoyl-ethyl)-2-(2,2-dimethyl-butyryloxy)-phenyl ester was obtained as a white solid, 0.520 g.

### Step 2

### S)-2-[3,4-Bis-(2,2-dimethyl-butyryloxy)-phenyl]-1-carbamoyl-ethyl-ammonium chloride

2,2-Dimethyl-butyric acid 4-((S)-2-tert-butoxycarbonylamino-2-carbamoyl-ethyl)-2-(2,2-dimethyl-butyryloxy)-phenyl ester (0.480 g) was dissolved in dichloromethane (6 mL) cooled with an ice-bath. 4M HCl in dioxane (2 mL) was added. After removal of the solvent, the solid was washed with diethyl ether, (S)-2-[3,4-Bis-(2,2-dimethyl-butyryloxy)-phenyl]-1-carbamoyl-ethyl-ammonium chloride was obtained as a white solid (0.420 g). m/z 393 (MH⁺), 410 (MNH₄⁺), 415 (MNa⁺). ¹H NMR (400 MHz, DMSO-d₆) δ 0.91 (m, 6H), 1.24 (s, 6H), 1.25 (s, 6H), 1.66 (m, 4H), 3.02 (m, 1H), 3.15 (m, 1H), 3.99 (t, J=6.5 Hz, 1H), 7.15-7.23 (m, 3H), 7.60 (s, 1H), 7.99 (s, 1H), 8.25 (br, 3H).

### EXAMPLE 8

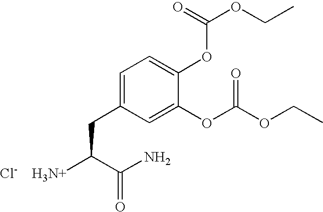

### (S)-2-(3,4-Bis-ethoxycarbonyloxy-phenyl)-1-carbamoyl-ethyl-ammonium chloride

### Step 1

### Carbonic acid 5-((S)-2-tert-butoxycarbonylamino-2-carbamoyl-ethyl)-2-ethoxycarbonyloxy-phenyl ester ethylester

[(S)-1-Carbamoyl-2-(3,4-dihydroxy-phenyl)-ethyl]-carbamic acid tert-butyl ester (402 mg) was dissolved in a mixture of DMA (2 ml) and dichloromethane (4 ml). Ethyl chloroformate (0.32 ml) was added followed by triethylamine (0.47 ml) and the mixture stirred and heated at 50 C overnight.

The mixture was diluted with dichloromethane and washed with water. Drying (MgSO₄) and evaporation gave a solid which was triturated with ethyl acetate-hexane. Carbonic acid 5-((S)-2-tert-butoxycarbonylamino-2-carbamoyl-ethyl)-2-ethoxycarbonyloxy-phenyl ester ethylester was obtained as a white solid, 367 mg, HPLC/MS (Method A) retention time 5.59 min, m/z 441 (MH⁺), 458 (MNH₄⁺).

### Step 2

### (S)-2-(3,4-Bis-ethoxycarbonyloxy-phenyl)-1-carbamoyl-ethyl-ammonium chloride

Carbonic acid 5-((S)-2-tert-butoxycarbonylamino-2-carbamoyl-ethyl)-2-ethoxycarbonyloxy-phenyl ester ethylester (362 mg) was dissolved in 4M HCl-dioxane (5 ml) at room temperature. After approximately 1.5 hr evaporation of solvent gave a white solid which was triturated with hexane to give (S)-2-(3,4-Bis-ethoxycarbonyloxy-phenyl)-1-carbamoyl-ethyl-ammonium chloride, 306 mg; NMR (500 MHz, d₆ DMSO) 1.28 (3H, t J 7), 1.29 (3H, t J 7), 3.04 (1H, dd J 14, 7.5), 3.15 (1H, dd J 14, 6), 4.00 (1H, dd J 7.5, 6), 4.25 and 4.26 (together 4H, two q J 7), 7.25 (1H, dd J 8.5, 2), 7.32 (1H, d J 2), 7.38 (1H, d J 8.5), 7.59 (1H, br s slow exch D₂O), 8.00 (1H, br s slow exch D₂O), 8.24 (3H, br s exch D₂O); HPLC/MS (Method A) retention time 3.26 min, m/z 341 (MH⁺).

### EXAMPLE 9

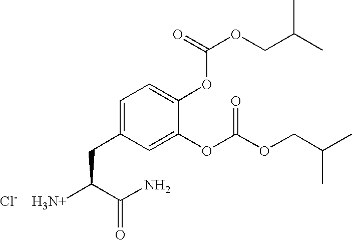

### (S)-2-(3,4-Bis-isobutoxycarbonvioxy-Phenyl)-1-carbamoyl-ethyl-ammonium chloride

### Step 1

### Carbonic acid 5-((S)-2-tert-butoxycarbonylamino-2-carbamoyl-ethyl)-2-isobutoxycarbonyloxy-phenyl ester isobutyl ester

[(S)-1-Carbamoyl-2-(3,4-dihydroxy-phenyl)-ethyl]-carbamic acid tert-butyl ester (400 mg) was dissolved in a mixture of DMA (2 ml) and dichloromethane (4 ml). Isobutyl chloroformate (0.43 ml) was added dropwise with stirring followed by triethylamine (0.46 ml) and a catalytic amount of DMAP. The mixture was stirred and heated at 50 C overnight.

The mixture was diluted with dichloromethane and water and the organic phase washed with dil HCl, aq sodium bicarbonate and brine. Drying (MgSO₄) and evaporation gave a solid which was triturated with ether-hexane (ca 1:1) to give carbonic acid 5-((S)-2-tert-butoxycarbonylamino-2-carbamoyl-ethyl)-2-isobutoxycarbonyloxy-phenyl ester isobutyl ester

as a white solid 477 mg, HPLC/MS (Method A) retention time 6.97 min, m/z 497 (MH⁺), 514 (MNH₄⁺).

### Step 2

### (S)-2-(3,4-Bis-isobutoxycarbonyloxy-phenyl)-1-carbamoyl-ethyl-ammonium chloride

5-((S)-2-tert-butoxycarbonylamino-2-carbamoyl-ethyl)-2-isobutoxycarbonyloxy-phenyl ester isobutyl ester (463 mg) was dissolved in 4M HCl-dioxane (10 ml). After approximately 1.5 hr evaporation of solvent gave an amorphous white solid which was triturated with hexane and vacuum dried at ca 45 C. (S)-2-(3,4-Bisisobutoxycarbonyloxy-phenyl)-1-carbamoyl-ethyl-ammonium chloride was obtained as a white solid, 377 mg, NMR (500 MHz, d₆ DMSO) 0.92 and 0.93 (together 12H, two d J 6.5), 1.96 and 1.97 (together 2H, two sept J 6.5), 4.0-4.2 (5H, m), 7.26 (1H, dd J 8, 2), 7.33 (1H, d J 2), 7.38 (1H, d J 8), 7.59 (1H, br s exch D₂O), 8.00 (1H, br s exch D₂O), 8.25 (ca 3H, br s exch D₂O);); HPLC/MS (Method A) retention time 4.65 min, m/z 397 (MH⁺).

### EXAMPLE 10

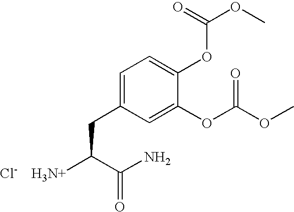

### (S)-2-(3,4-Bis-methoxycarbonvioxy-phenyl)-1-carbamoyl-ethyl-ammonium chloride

### Step 1

### Carbonic acid 5-((S)-2-tert-butoxycarbonylamino-2-carbamoyl-ethyl)-2-methoxycarbonyloxy-phenyl ester methyl ester

[(S)-1-Carbamoyl-2-(3,4-dihydroxy-phenyl)-ethyl]-carbamic acid tert-butyl ester (355 mg) was dissolved in a mixture of DMA (2 ml) and dichloromethane (4 ml) under nitrogen. Methyl chloroformate (0.24 ml) was added dropwise with stirring followed by triethylamine (0.42 ml). The mixture was stirred at room temperature for 2 hours when HPLC showed no starting material. The mixture was diluted with dichloromethane and washed with water. Drying (MgSO₄) and evaporation gave an oil which solidified on addition of water (15 ml). Trituration with ether-hexane (1:1) gave carbonic acid 5-((S)-2-tert-butoxycarbonylamino-2-carbamoyl-ethyl)-2-methoxycarbonyloxy-phenyl ester methyl ester

as a white solid, 402 mg, HPLC/MS (Method A) retention time 4.98 min, m/z 413 (MH⁺), 430 (MNH₄⁺).

### Step 2

### (S)-2-(3,4-Bis-methoxycarbonyloxy-phenyl)-1-carbamoyl-ethyl-ammonium chloride

Carbonic acid 5-((S)-2-tert-butoxycarbonylamino-2-carbamoyl-ethyl)-2-methoxycarbonyloxy-phenyl ester methyl ester (402 mg) was dissolved in 4M HCl-dioxane (10 ml) at room temperature. After 1.5 hours the solution was evaporated to dryness. Trituration with ether gave (S)-2-(3,4-Bis-methoxycarbonyloxy-phenyl)-1-carbamoyl-ethyl-ammonium chloride as a white solid, 288 mg, NMR (500 MHz, d₆ DMSO)), 3.05 (1H, dd, J 14, 7.5), 3.16 (1H, dd, J 14, 6), 3.84 (3H, s), 3.85 (3H, s), 4.00 (1H, dd J 7.5, 6), 7.27 (1H, dd J 8, 2), 7.34 (1H, d J 2), 7.39 (1H, d J 8), 7.59 (1H, br s, exch D20), 8.02 (1H, br s exch D₂O), 8.27 (3H, br s exch D₂O); Electrospray MS 313 (MH⁺).

### EXAMPLE 11

### 13PE1 and 13PE3

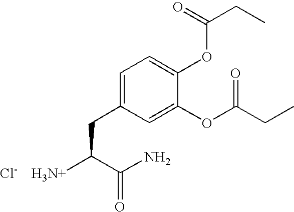

### (S)-2-(3,4-Bis-propionyloxy-phenyl)-1-carbamoyl-ethyl-ammonium chloride

### Step 1

### Propionic acid 4-((S)-2-tert-butoxycarbonylamino-2-carbamoyl-ethyl)-2-propionyloxy-phenyl ester

[(S)-1-Carbamoyl-2-(3,4-dihydroxy-phenyl)-ethyl]-carbamic acid tert-butyl ester (355 mg) was dissolved in a mixture of DMA (2 ml) and dichloromethane (7 ml). Propionyl chloride (0.32 ml) was added followed by triethylamine (0.5 ml) and the mixture was stirred and heated at 50 C overnight.

The mixture was diluted with dichloromethane and washed with dil HCl, aq sodium bicarbonate and brine. Drying (MgSO₄) and evaporation gave a solid which was recrystallised from ethyl acetate to give propionic acid 4-((S)-2-tert-butoxycarbonylamino-2-carbamoyl-ethyl)-2-propionyloxy-phenyl ester as a white solid 167 mg, HPLC/MS (Method A) retention time 5.53 min, m/z 409 (MH⁺), 426 (MNH₄⁺).

### (S)-2-(3,4-Bis-propionyloxy-phenyl)-1-carbamoyl-ethyl-ammonium chloride

Propionic acid 4-((S)-2-tert-butoxycarbonylamino-2-carbamoyl-ethyl)-2-propionyloxy-phenyl ester (166 mg) was dissolved in 4M HCl-dioxane (4 ml). After 1 hour at room temperature a light precipitate had begun to form. After a total of ca 1.5 hours ether (4 ml) was added to the mixture and the solid was filtered off and washed with more ether. (S)-2-(3,4-Bis-propionyloxy-phenyl)-1-carbamoyl-ethyl-ammonium chloride was obtained as a white solid, 118 mg, NMR (500 MHz, d₆ DMSO) 1.11 and 1.12 (together 6H, two t J 7.5), 2.57 and 2.59 (together 2H, two q J 7.5), 3.03 (1H, dd, J 14, 6.5), 3.12 (1H, dd, J 14, 6), 3.98 (1H, dd, J 6.5, 6), 7.19-7.24 (3H, m, shows d and two dd in D₂O), 7.58 (1H, br s, exch D₂O), 7.98 (1H, br s exch D20), 8.25 (3H, br s exch D₂O); Electrospray MS 309 (MH⁺).

### EXAMPLE 12

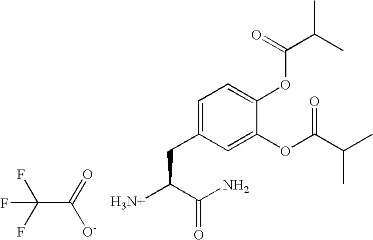

### (S)-2-(3,4-Bis-isobutyryloxy-phenyl)-1-carbamoyl-ethyl-ammonium trifluoroacetate

### Step 1

### Isobutyric acid 4-((S)-2-tert-butoxycarbonylamino-2-carbamoyl-ethyl)-2-isobutyryloxy-phenyl ester

[(S)-1-Carbamoyl-2-(3,4-dihydroxy-phenyl)-ethyl]-carbamic acid tert-butyl ester (376 mg) was treated with isobutyryl chloride and triethylamine in DMA as described in example 2 step 1. Excess acid chloride and base were added and the mixture was heated to 70 C overnight in order to complete the reaction. Isobutyric acid 4-((S)-2-tert-butoxycarbonylamino-2-carbamoyl-ethyl)-2-isobutyryloxy-phenyl ester was obtained as a white solid, 105 mg; HPLC/MS (Method A) retention time 6.25 min, m/z 437 (MH⁺), 454 (MNH₄⁺).

### Step 2

### (S)-2-(3,4-Bis-isobutyryloxy-phenyl)-1-carbamoyl-ethyl-ammonium chloride

Isobutyric acid 4-((S)-2-tert-butoxycarbonylamino-2-carbamoyl-ethyl)-2-isobutyryloxy-phenyl ester (105 mg) was dissolved in 4M HCl-dioxane (3 ml) at room temperature. After approximately 1.5 hr solvent was evaporated to give a gum that was triturated with ether to give (S)-2-(3,4-Bis-isobutyryloxy-phenyl)-1-carbamoyl-ethyl-ammonium chloride as a white solid, 85 mg; NMR (500 MHz, d₆ DMSO) 1.20 and 1.22 (together 12H, two d J ca 7), 2.78 and 2.80 (together 2H, two sept J ca 7), 3.02 (1H, dd J 14, 7.5), 3.12 (1H, dd J 14, 6) 3.98 (1H, dd J 7.5, 6), 7.18-7.20 (2H, m, shows d J 2 and dd J 8, 2 in D₂O), 7.23 (1H, d J 8), 7.60 (1H, br s), 7.94 (1H, br s), 8.21 (3H, br s exch D₂O); HPLC/MS (Method A) retention time 3.70 min, m/z 337 (MH⁺).

### Step 3

### (S)-2-(3,4-Bis-isobutyryloxy-phenyl)-1-carbamoyl-ethyl-ammonium trifluoroacetate

(S)-2-(3,4-Bis-isobutyryloxy-phenyl)-1-carbamoyl-ethyl-ammonium chloride (310 mg) was suspended in dichloromethane (3 ml) and trifluoroacetic acid (2 ml) was added. After approximately 1.5 hr solvent was evaporated to give a gum that was triturated with hexane to give (S)-2-(3,4-Bis-isobutyryloxy-phenyl)-1-carbamoyl-ethyl-ammonium trifluoroacetate as a white solid, 350 mg; NMR (500 MHz, d₆ DMSO) 1.20 and 1.22 (together 12H, two d J 7), 2.79 and 2.80 (together 2H, two sept J 7), 2.98 (1H, dd J 14, 8), 3.10 (1H, dd J 14, 5.5), 3.97 (1H, br m), 7.17-7.19 (2H, m, shows d J 2 and dd J 8, 2 in D₂O), 7.24 (1H, d J 8), 7.61 (1H, br s exch D₂O), 7.87 (1H, br s exch D₂O), 8.14 (3H, br s exch D₂O); HPLC/MS (Method A) retention time 3.76 min, m/z 337 (MH⁺).

### Biological Results

Compounds of the examples above were tested in the following animal model of dopamine deficiency behaviour:

### Assessment of Activity in 6-OHDA-Lesioned Rats

Animals Male Wistar rats, 250 g, Harlan Ltd.
Housing Animals were housed in groups of 4 on a 12-h light-dark cycle with an environment of 50% humidity and temperature of 21±2° C. in accordance with Animals (Scientific Procedures) Act 1996 Home Office regulations. Rats had access to food and water ad libitum.
Licence All animals used in this study were treated in accordance with the UK 1986 Animals (Scientific Procedures Act).
Procedure
Surgery Male Wistar rats were treated with desipramine (25 mg/kg ip, 30 minutes prior to 6-OHDA) to protect noradrenergic terminals. Rats were then anaesthetized in an induction chamber using isofluorane (1-2% in 95% O₂, 5% CO₂ carrier gas), placed in a Kopf stereotaxic frame and anaesthesia maintained with 0.5-1.0% isofluorane. An incision was made in the scalp and a 0.8-mm-diameter hole made in the skull at coordinates AP: −0.26 mm L: +2.0 mm (all coordinated measured from bregma). The neurotoxin 6-hydroxydopamine (6-OHDA) (8 μg free base in 4 μL of 0.9% saline containing 0.05% ascorbic acid) was injected into the left median forebrain bundle at a constant rate over 4 min (1 μl/min) using a 10-μL Hamilton syringe lowered to −8 mm below the dura. The needle remained in place for a further 4 min before being removed, and the wound cleaned and sutured. Flunixin hydrochloride (2.5 mg/kg, Dunlop's Veterinary Supplies, Dumfries, UK) was administered for pain relief and a rehydration treatment of 5% glucose in 0.9% saline (up to 5 ml ip) was given prior to recovery from the anaesthetic.
Behavioural Assessment
Confirmation of the lesion
   At least 2 weeks following surgery, animals were examined for rotational behaviour (see below) in response to the administration of apomorphine hydrochloride (0.5 mg/kg s.c. in 0.9% saline containing 0.05% ascorbic acid) to evaluate the extent of the lesion. Only those rats exhibiting >6 turns/min at peak activity were used in future studies.
Assessment of the Induction of Rotational Activity by Test Compounds
   At least 1 week after apomorphine administration, rats (n=4-8 per treatment) were tested for rotational activity with either a test drug or L-DOPA. These were administered either via the intraperitoneal (ip) route or orally by gavage (po). Animals were treated with benserazide (10 mg/kg) and placed in rotometers (Med Associates) for up to 30 min to measure basal activity. They were then treated with test compound or L-DOPA (63.4 μmole/kg ip or po). Rotation behaviour was assessed for up to 4 hours after test drug/L-DOPA administration. Animals were typically treated with a series of compounds for comparative purposes. Each treatment was administered at least 1 day apart.
Data Analysis
   The number of rotations measured per 10 minutes over the 4 hour period was determined. Animals were considered active if they turned >10 turns per 10 minutes. From this data the following parameters were measured:
      A Total activity (AUC activity, where AUC=area under the locomotor-activity/time curve)
      B Peak activity
      C Duration of activity
   Values are quoted as % of L-DOPA induced effects.

By way of example, the compound of Example 5 above, administered p.o., showed an AUC which was 89% of that of L-Dopa, a peak activity which was 118% of that of L-Dopa, and a duration of activity which was 71% of that of L-Dopa.

1. A compound of formula (I) or a salt, hydrate or solvate thereof: 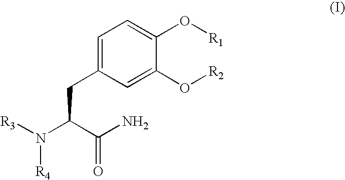
wherein:
R₁ and R₂ are independently selected from —C(═O)R₅ or —C(═O)OR₅; or one of R₁ and R₂ is hydrogen and the other is —C(═O)R₅ or —C(═O)OR₅;
R₃ and R₄ are independently selected from
hydrogen,
optionally substituted C₁-C₆ alkyl, C₃-C₆ cycloalkyl, C₂-C₆ alkenyl, or C₂-C₆
alkynyl,
—CH₂Q,
—C(═O)R₅,
—C(═O)OR₅,
—C(═O)NR₅R₆, or
R₅ is hydrogen or optionally substituted C₁-C₆ alkyl or —CH₂Q;
R₆ is hydrogen or optionally substituted C₁-C₆ alkyl or —CH₂Q; and
Q is an optionally substituted monocyclic carbocyclic or heterocyclyl ring of 3 to 6 ring atoms;
PROVIDED THAT when R₁ and R₂ are each —C(═O)R₅ wherein R₅ is methyl, and R₃ is hydrogen, then R₄ is not tert-butoxycarbonyl.

2. A compound as claimed in claim 1 wherein one of R₁ and R₂ is hydrogen.

3. A compound as claimed in claim 1 wherein R₁ and R₂ when not hydrogen are independently —C(═O)R₅ wherein R₅ is methyl.

4. A compound as claimed in claim 3 wherein R₁ and R₂ are the same.

5. A compound as claimed in claim 1 wherein R₃ and R₄ are both hydrogen.

6. A compound as claimed in claim 1 wherein R₃ and R₄, when not hydrogen, are independently selected from methyl, ethyl, allyl, benzyl, acetyl, phenylcarbonyl, phenoxycarbonyl or aminocarbonyl.

7. A compound as claimed in claim 1 wherein any optional substituents are selected from methyl, trifluoromethyl, methoxy, trifluoromethoxy, cyclopropyl, halogen, cyano, hydroxy, mercapto, oxo, —NH₂, —NHR^{A} or —NR^{A}R^{B} wherein R^{A} and R^{B} are independently methyl or ethyl.

8. A compound as claimed in claim 1 wherein R₁ and R₂ are each acetyl or (4-methylphenyl)-carbonyl, and R₃ and R₄ are both hydrogen.

9. A pharmaceutical composition comprising a compound as claimed in claim 1 together with a pharmaceutically acceptable carrier.

10. (canceled)

11. A method of treatment of a condition associated with impaired dopaminergic signalling in a subject, comprising administrating to the subject an amount of a compound as claimed in claim 1, or the compound defined in claim 1 wherein R₁ and R₂ are each —C(═O)R₅ wherein R₅ is methyl, and R₃ is hydrogen, and R₄ is tert-butoxycarbonyl, effective to reduce such impairment of dopaminergic signalling.

12. The method as claimed in claim 11, wherein the condition is Parkinson's disease, or Restless Legs Syndrome

13. The method as claimed in claim 11, wherein the condition is Tourette's syndrome, attention deficit hyperactive disorder, generation of pituitary tumours, a Parkinson-plus syndrome, levodopa responsive dystonia, dyskinesia, periodic movements in sleep, dysphagia or neuroleptic malignant syndrome.

14. A method of treatment of a condition associated with impaired dopaminergic signalling in a subject, comprising administrating to the subject an amount of a compound as claimed in claim 1 wherein R₁ and R₂ are each —C(═O)R₅ wherein R₅ is methyl, and R₃ is hydrogen, and R₄ is tert-butoxycarbonyl, effective to reduce such impairment of dopaminergic signalling.
